(19) United States
(12) Patent Application Publication
(10) Pub. No.: US 2022/0118184 A1
(43) Pub. Date: Apr. 21, 2022

(54) A SHIELD TRIGGER MECHANISM AND AN INJECTION DEVICE WITH A SHIELD TRIGGER MECHANISM

(71) Applicant: Novo Nordisk A/S, Bagsvaerd (DK)

(72) Inventors: Nikolaj Thorsoe Kronborg, Koebenhavn N (DK); Simon Munch Pedersen, Koebenhavn N (DK)

(21) Appl. No.: 17/419,453

(22) PCT Filed: Dec. 10, 2019
(86) PCT No.: PCT/EP2019/084326

§ 371 Date: Jun. 29, 2021

(30) Foreign Application Priority Data
Jan. 4, 2019 (EP) 19150379.6

(51) Int. Cl. A61M 5/20; A61M 5/315; A61M 5/32

(52) U.S. Cl. CPC A61M 5/2033; A61M 5/31553; A61M 2005/2013; A61M 5/31535; A61M 5/3202

(57) ABSTRACT

The invention relates to a spring driven injection device for expelling doses a liquid drug. A housing structure secures a container containing a liquid drug to be injected via a spring driven dose engine. A needle shield is rotatably relatively to the housing structure between a locked position and an unlocked position. In the locked position the needle shield is prevented from axial movement and in the unlocked position axially movement is possible by applying an axial force onto the needle shield. Axial movement of the needle shield activates the spring driven dose engine to automatically eject the dose of the liquid drug from the container. The needle shield is rotational guided from the locked position to the unlocked position in a track arrangement which is further configured to prevent the needle shield in rotation from the locked position to the unlocked position during application of the axial force to the needle shield.

### THE TECHNICAL FIELD OF THE INVENTION

The invention relates to a shield trigger mechanism for triggering the ejection of a dose of a liquid drug from an automatic spring driven injection mechanism. The invention especially relates to such shield trigger mechanism wherein the spring driven injection device is triggered by an axial movement of the needle shield.

The invention further relates to a spring driven injection device with a shield trigger mechanism such that the spring is released to eject the dose in response to an axial force being applied onto the needle shield.

### DESCRIPTION OF RELATED ART

Spring driven injection devices for the automatic injection of doses of a liquid drug are widely known. A large group of such spring driven injection devices are based on a torsion spring driving the injection.

The general principle of such torsion spring driven injection devices are that the dose of liquid drug to be injected is forced out from the injection device by the torque of a torsion spring. The torque of the torsion spring is usable build up during setting of the size of the dose to be ejected by rotation of a dose setting element which is usually rotated relatively to a housing structure. Alternatively, the torque is stored in the torsion spring by the manufacturer of the injection device. During injection the torque stored in the torsion spring is, at least partly, released to drive out the set dose from the drug container. In order to release the torque of the torsion spring, the user needs to operate an activation mechanism which thus triggers the spring driven injection device to deliver the set dose.

WO 2006/126902 and WO 2006/076921 disclose different examples of such activation mechanisms.

In WO 2006/126902 the release of the torque is activated by the user operating a sliding button which is physically provided on the outer surface of the housing of the pen shaped injection device. By pushing this sliding button along the surface of the housing structure in the distal direction the torque stored in the torsion spring is released to drive the piston rod forward to thereby expel the set dose.

In a different torsion spring driven injection device disclosed in WO 2006/076921, the release of the torque is done by activation of an injection button provided at the most proximal end of the injection device.

It is further generally known from other injection devices to hide the needle cannula during injection by a telescopically movable needle shield such that the user is enabled to perform the full injection without visually having eye contact with the needle cannula. The ability to perform an injection without actually seeing the needle is very helpful for people who suffer from anxiety regarding injection needles.

When such telescopically needle shield is used in a torsion spring driven injection device it is known to use the needle shield to releases the torque stored in the torsion spring. An example of such injection device is provided in WO 2017/032599.

However, when the needle shield is used to trigger the release of the torsion spring it is important that the user cannot accidentally move the needle shield proximally and thus release the torque stored in the torsion spring.

In WO 2017/032599 this is solved by making the needle shield able to rotate between a locked and an unlocked position such that the user is required to rotate the needle shield before it can be moved proximally to release the torque of the torsion spring.

The same is the scenario in PCT application PCT/EP2019/065451 wherein the needle shield during rotation is guided in a track structure provided in the housing structure. This guiding structure guides the needle shield from a first locked position to second unlocked position. The track arrangement is preferably shaped such that the needle shield moves helically during rotation.

However, when using the injection device disclosed in PCT application PCT/EP2019/065451 users are able to push the needle shield against the skin to perform an injection without first rotating the needle shield i.e. with the needle shield in the locked position. The result of this is that no dose is expelled. When the user realizes this, the user can be inclined to rotate the needle shield while maintaining the needle shield pressed against the skin. This has the unfortunate effect that once the needle shield enters into the unlocked position both the needle insertion into the skin and the release of the torque of the torsion spring and thus the injection happens very suddenly. This sometimes comes as a surprise to the user which then accidentally removes the needle shield from the skin and consequently also the injection needle from the skin thereby potentially spilling liquid drug.

### DESCRIPTION OF THE INVENTION

It is henceforth an object of the present invention to provide a shield trigger mechanism which ensures that the user is properly guided through the process of performing the injection correctly preferably such that liquid drug is not spilled due to a wrongful handling of the spring driven injection.

It is further an object of the present invention to provide a spring driven injection device wherein the trigger mechanism helps and supports the user through the injection process.

Accordingly, in one aspect of the present invention, a shield trigger mechanism is provided which is suitable for triggering the ejection of a dose of a liquid drug from a spring driven injection device.

The shield trigger mechanism comprises a needle shield which is rotatably relatively to a housing structure between a locked position and an unlocked position.
In the locked position the needle shield is prevented from axial movement relatively to the housing structure, and
in the unlocked position the needle shield is axially movable relatively to the housing structure in response to an axial force being applied onto the needle shield to thereby activate the spring driven injection device to automatically eject the dose of the liquid drug.

The axial movement of the needle shield which is possible after the needle shield has been rotated into the unlocked position activates the spring driven injection device to automatically eject the dose of the liquid drug.

Further, the needle shield is rotational guided from the locked position to the unlocked position by a track arrangement which is configured to prevent the needle shield in rotation from the locked position to the unlocked position during application of the axial force to the needle shield. Preferably a physical stop is incorporated into the track arrangement.

The configuration of the physical stop in the track arrangement henceforth prevent that a user can simultaneously press the needle shield against the skin and rotate the needle shield into the unlocked position such that an injection is being performed. Due to the physical stop of the track configuration the user needs to perform the two actions of pressing the needle shield against the skin and rotating the needle shield sequentially and not simultaneously.

The track arrangement is preferably either radial or helical. In one example, the track arrangement comprises a helical track region such that the resulting movement occurring when the user rotate the needle shield is a helical movement.

The helical track region can either be associated with the needle shield or with the housing structure. In an example wherein the helical track region is provided in the housing structure, the needle shield is preferably provided with a protrusion which is guided in the helical track region of the housing structure.

The tracks, track regions and protrusions discussed herein are preferably provided in pairs but can obviously be provided in any random number without deviating from the principle of the present invention.

Rotation of the needle shield thus makes the protrusion on the needle shield which preferably points outwardly in a radial direction move through the helical track region in a helical movement thus making the needle shield perform a helical movement preferably in the proximal direction.

In a further example, the helical track region terminates into an axial track such that once the protrusion on the needle shield comes to the end of the helical track region, the protrusion is automatically situated in the distal part of the axial track. The axial track is the track formation which allows the protrusion and thus the needle shield to move axially in the proximal direction and to trigger the injection. The position of the protrusion in the area wherein the helical track region terminates into the axial track is the position in which the needle shield is unlocked and free to move in the proximal direction during injection.

In the area where the helical track region and the axial track meet the physical stop is preferably provided which prevents the protrusion on the needle shield from moving from the helical track region and into the axial track during application of the axial force to the needle shield. The physical stop which in one example can be a physical knop, a flange, a ridge or any similar obstacle provided on a side wall of either of the tracks prevents the protrusion from passing the physical stop as long as the protrusion is pressed against the proximal side wall of the track. In one preferred example, the physical stop is built into the proximal side of the helical (second) track region. As the protrusion is provided radially on the needle shield this means that as long as a force is being applied onto the needle shield pushing the needle shield in the proximal direction and thus the protrusion against the side wall, this physical stop prevents the protrusion from being rotated pass the physical stop and into the axial track.

However, when the force is removed from the needle shield, the needle shield is urged in the distal direction such that the protrusion is able to escape the physical stop.

A compression force is applied onto the needle shield in the distal direction which compression moves the needle shield towards its initial position following an injection. The compression force thus also moves the protrusion distally to rest against the distal wall side of the helical track region when the user removes the needle shield from the skin.

In a second aspect the invention relates to an injection device and preferably a torsion spring driven automatic injection device which comprises:

A housing structure securing a container such as a cartridge which contains a liquid drug to be injected and which housing structure further holds a spring driven dose engine, preferably a torsion spring operated dose engine.

A needle shield, which is rotatably relatively to the housing structure between a locked position and an unlocked position;
In the locked position the needle shield is prevented from axial movement relatively to the housing structure, and
in the unlocked position the needle shield is axially movable relatively to the housing structure in response to an axial force being applied onto the needle shield to thereby activate the spring driven dose engine to automatically eject the dose of the liquid drug.

The axial movement of the needle shield which is possible after the needle shield has been rotated into the unlocked position activates the spring driven injection device to automatically eject the dose of the liquid drug.

Further, the needle shield is rotational guided from the locked position to the unlocked position by a track arrangement which is configured to prevent the needle shield in rotation from the locked position to the unlocked position during application of the axial force to the needle shield. Preferably, a physical stop is incorporated into the track arrangement.

The injection device is preferably a pre-filled injection device as further defined in the present. This means that the cartridge is permanently embedded in the housing structure.

The features of the first embodiment are also applicable to the second embodiment.

Thus, the track arrangement comprises a helical track region.

Further, the helical track region is preferably associated with the housing structure.

The needle shield is provided with a protrusion guided in the helical track region. The protrusion is preferably a radial protrusion pointing in the outwardly direction.

The helical track region preferably terminates into an axial track which guides the protrusion during injection.

The junction between the helical track region and the axial track are preferably provided with the physical stop preventing the protrusion on the needle shield in moving from the helical track region and into the axial track as long as the axial force is being applied on to the needle shield.

Once the axial force is removed, the the needle shield is urged in the distal direction preferably by an axial force working in the distal direction such that the protrusion is able to escape the physical stop.

### Definitions

An “injection pen” is typically an injection apparatus having an oblong or elongated shape somewhat like a pen for writing. Although such pens usually have a tubular cross-section, they could easily have a different cross-section such as triangular, rectangular or square or any variation around these geometries.

The term “Needle Cannula” is used to describe the actual conduit performing the penetration of the skin during injection. A needle cannula is usually made from a metallic material such as e.g. stainless steel and preferably connected to a hub made from a suitable material e.g. a polymer. A needle cannula could however also be made from a polymeric material or a glass material.

As used herein, the term “Liquid drug” is meant to encompass any drug-containing flowable medicine capable of being passed through a delivery means such as a hollow needle cannula in a controlled manner, such as a liquid, solution, gel or fine suspension. Representative drugs includes pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form.

“Cartridge” is the term used to describe the container actually containing the drug. Cartridges are usually made from glass but could also be moulded from any suitable polymer. A cartridge or ampoule is preferably sealed at one end by a pierceable membrane referred to as the “septum” which can be pierced e.g. by the non-patient end of a needle cannula. Such septum is usually self-sealing which means that the opening created during penetration seals automatically by the inherent resiliency once the needle cannula is removed from the septum. The opposite end of the cartridge is typically closed by a plunger or piston made from rubber or a suitable polymer. The plunger or piston can be slidable moved inside the cartridge. The space between the pierceable membrane and the movable plunger holds the drug which is pressed out as the plunger decreased the volume of the space holding the drug.

The cartridges used for both pre-filled injection devices and for durable injections devices are typically factory filled by the manufacturer with a predetermined volume of a liquid drug. A large number of the cartridges currently available contains either 1.5 ml or 3 ml of liquid drug.

Since a cartridge usually has a narrower distal neck portion into which the plunger cannot be moved not all of the liquid drug contained inside the cartridge can actually be expelled. The term “initial quantum” or “substantially used” therefore refers to the injectable content contained in the cartridge and thus not necessarily to the entire content.

By the term “Pre-filled” injection device is meant an injection device in which the cartridge containing the liquid drug is permanently embedded in the injection device such that it cannot be removed without permanent destruction of the injection device. Once the pre-filled amount of liquid drug in the cartridge is used, the user normally discards the entire injection device. Usually the cartridge which has been filled by the manufacturer with a specific amount of liquid drug is secured in a cartridge holder which is then permanently connected in a housing structure such that the cartridge cannot be exchanged.

This is in opposition to a “Durable” injection device in which the user can himself change the cartridge containing the liquid drug whenever it is empty. Pre-filled injection devices are usually sold in packages containing more than one injection device whereas durable injection devices are usually sold one at a time. When using pre-filled injection devices an average user might require as many as 50 to 100 injection devices per year whereas when using durable injection devices one single injection device could last for several years, however, the average user would require 50 to 100 new cartridges per year.

Using the term “Automatic” in conjunction with injection device means that, the injection device is able to perform the injection without the user of the injection device delivering the force needed to expel the drug during dosing. The force is typically delivered—automatically—by an electric motor or by a spring drive. The spring for the spring drive is usually strained by the user during dose setting, however, such springs are usually prestrained in order to avoid problems of delivering very small doses. Alternatively, the spring can be fully preloaded by the manufacturer with a preload sufficient to empty the entire drug cartridge though a number of doses. Typically, the user activates a latch mechanism provided either on the surface of the housing or at the proximal end of the injection device to release—fully or partially—the force accumulated in the spring when carrying out the injection.

The term “Permanently connected” or “permanently embedded” as used in this description is intended to mean that the parts, which in this application is embodied as a cartridge permanently embedded in the housing, requires the use of tools in order to be separated and should the parts be separated it would permanently damage at least one of the parts.

All references, including publications, patent applications, and patents, cited herein are incorporated by reference in their entirety and to the same extent as if each reference were individually and specifically indicated to be incorporated by reference and were set forth in its entirety herein.

All headings and sub-headings are used herein for convenience only and should not be constructed as limiting the invention in any way.

The use of any and all examples, or exemplary language (e.g. such as) provided herein, is intended merely to better illuminate the invention and does not pose a limitation on the scope of the invention unless otherwise claimed. No language in the specification should be construed as indicating any non-claimed element as essential to the practice of the invention. The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### BRIEF DESCRIPTION OF THE DRAWINGS

The invention will be explained more fully below in connection with a preferred embodiment and with reference to the drawings in which:

FIG. 1 show a perspective view of the injection device with the protective cap attached.

FIG. 2 show a perspective view of the injection device with the protective cap removed.

FIG. 3 show an exploded view of the housing structure together with the cartridge.

FIG. 4 show a cross-sectional view of the protective cap.

FIG. 5 show a cross-sectional view of the needle shield.

FIG. 6A show a perspective view of the injection device with the base part of the housing structure visually removed and the protective cap attached.

FIG. 6B show a perspective view of the injection device with the base part of the housing structure visually removed and the protective cap removed.

FIG. 7A show the engagement between the needle shield and the transfer element in the stop position.

FIG. 7B show the engagement between the needle shield and the transfer element in the relaxed position.

FIG. 7C show the engagement between the needle shield and the transfer element in the injection position.

FIG. 8A show a schematic view of the movement without the stop functionality and with no force applied onto the needle shield.

FIG. 8B show a schematic view of the movement without the stop functionality and with a force applied onto the needle shield.

FIG. 8C show a schematic view of the movement with the stop functionality and with a force applied onto the needle shield

The figures are schematic and simplified for clarity, and they just show details, which are essential to the understanding of the invention, while other details are left out. Throughout, the same reference numerals are used for identical or corresponding parts.

### DETAILED DESCRIPTION OF EMBODIMENT

When in the following terms as “upper” and “lower”, “right” and “left”, “horizontal” and “vertical”, “clockwise” and “counter clockwise” or similar relative expressions are used, these only refer to the appended figures and not to an actual situation of use. The shown figures are schematic representations for which reason the configuration of the different structures as well as their relative dimensions are intended to serve illustrative purposes only.

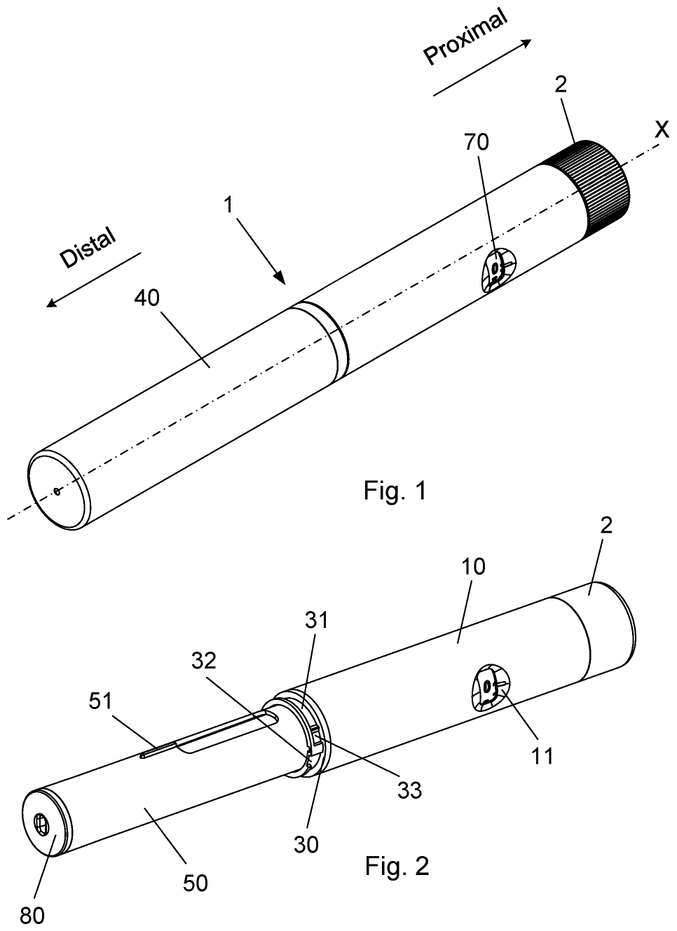

In that context it may be convenient to define that the term “distal end” in the appended figures is meant to refer to the end of the injection device securing the needle cannula and pointing towards the user during injection, whereas the term “proximal end” is meant to refer to the opposite end usually carrying the dose dial button as depicted in FIG. 1. Distal and proximal is meant to be along an axial orientation extending along the longitudinal axis (X) of the injection device as also disclosed in FIG. 1.

FIG. 1 and FIG. 2 disclose the injection device with and without the protective cap 40 attached to the housing structure 1. The injection device comprises a housing structure 1 which can be made from any number of separate pieces connected together to form a full outer housing.

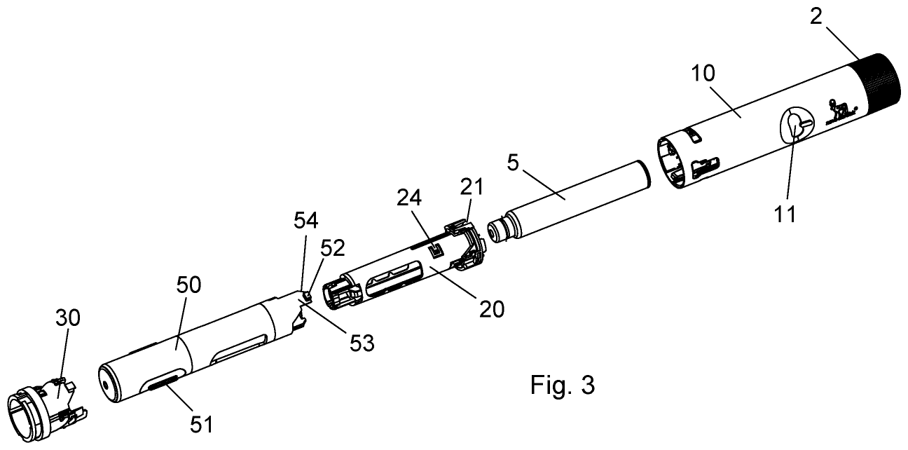

As disclosed in FIG. 3, the housing structure 1, in the present embodiment, comprises a base part 10, a cartridge holder part 20 and an initiator part 30 as also shown in PCT application PCT/EP2019/065451. These parts are preferably clicked together to form the housing structure 1.

The cartridge holder part 20 is in use covered by the movable needle shield 50 as also seen in FIG. 2. Internally the cartridge holder part 20 secures the cartridge 5 which contains the liquid drug to be injected. The base part 10 secures the dose engine which in the enclosed embodiment is a torsion spring driven dose engine as disclosed in WO 2019/002020.

When the housing structure 1 is assembled a helical track 60 emerges between the flanges of the cartridge holder part 20 and the initiator part 30 which helical track 60 locks around an outwardly pointing protrusion 52 on the needle shield 50 as will be explained. In the disclosed embodiment two such helical tracks 60 are provided. Each of the helical tracks 60 are functionally divided into two regions; a first track region 60A and a second track region 60B separated by a bridge 35 under which the outwardly pointing protrusions 52 are able to slide.

As there are two helical tracks 60 disclosed in this embodiment, the various other elements relating to these tracks 60 are preferably also provided in pairs. It is thus to be understood that even if described in singularity in the text, the various elements can be provided in plural.

The outwardly pointing protrusion 52 is provided on an axial extension 53 on the needle shield 50 as seen in FIG. 3 and in FIG. 5. The peripheral width of the outwardly pointing protrusion 52 is somewhat smaller than the peripheral width of this axial (and proximal) extension 53 of the needle shield 50. At one side the axial extension 53 is cut off in a sloped surface 54, the use of which will be explained later.

The protective cap 40 is mounted to cover the distal end of the housing structure 1 i.e. the cartridge holder part 20, while the opposite proximal end of the housing structure 1 i.e. the base part 10 is provided with a rotatable dose dial 2 which a user can rotate in a first rotational direction in order to set the size of the dose to be ejected. Since the injection device disclosed is an automatic spring operated injection device, the dose dial 2 is rotatable connected to the housing structure 1 such that the dose dial 2 do not move axially during dose setting but are allowed to rotate in relation to the housing structure 1.

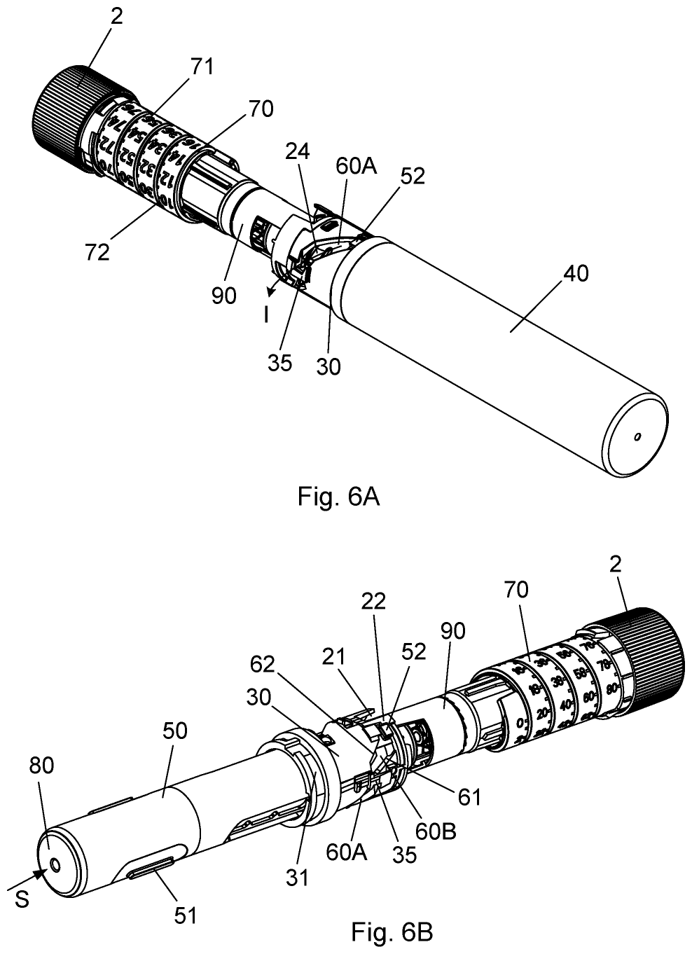

The base part 10 is further provided with a window 11 through which the user can inspect the rotatable scale drum 70 carrying indicia 71 indicating the size of the dose being set. As further seen in FIG. 6A-B, the rotatable scale drum 70 is externally provided with a helical track 72 engaging a similar thread segment provided on the inner surface of the base part 10 such that the scale drum 70 moves helically when rotated relatively to the housing structure 1 as it is commonly known from injection devices.

As best seen in FIG. 2, the initiator part 30 is distally provided with a peripheral track 31 which has at least one axial opening 32.

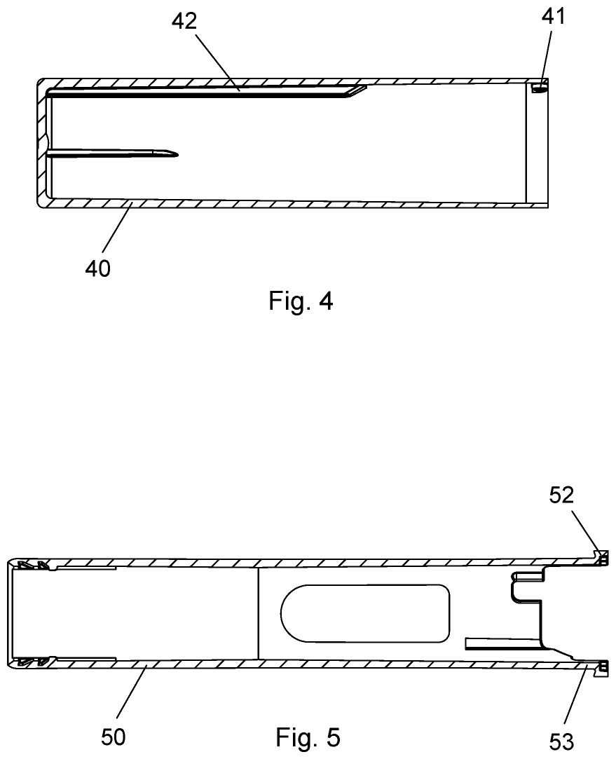

The protective cap 40 which is disclosed in a cross-sectional view in FIG. 4 is proximally and on the inner surface provided with an inwardly pointing protrusion 41 which engages the peripheral track 31 such that the user is required to rotate the protective cap 40 before it can be axially removed from the housing structure 1 by pulling the inwardly pointing protrusion 41 through the axial opening 32. Typically, there would be two such axial openings 32 to accommodate two inwardly pointing protrusions 41 such that the user is required to rotate the protective cap 40 a little less than 180° before the protective cap 40 can be axially removed. Further, as shown in FIG. 2, the peripheral track 31 can be equipped with a parking position 33 separated from the peripheral track 31 by an axial rib.

During this mandatory rotation of the protective cap 40, a longitudinal rib 42 also provided on the inner surface of the protective cap 40 engage a similar rib 51 (see e.g. FIG. 2) provided on the needle shield 50 which is thus forced to follow the rotation of the protective cap 40. In the disclosed embodiment two longitudinal ribs 42 and two ribs 51 are provided.

The needle shield 50 which is disclosed in a cross-sectional view in FIG. 5 is proximally provided with a number of outwardly pointing protrusions 52 which engages the helical tracks 60 provided between the cartridge holder part 20 and the initiator part 30 in the housing structure 1. Consequently, when the user rotates the protective cap 40 to remove it, this rotation is transferred to a similar rotation of the needle shield 50. Since the helical tracks 60 in the disclosed embodiment are in fact helical, the needle shield 50 translates helically in the proximal direction during rotation.

Distally the needle shield 50 carries a cleaning unit 80 which is secured to the needle shield 50 such that the cleaning unit 80 both rotate and move axially together with the needle shield 50, thus the cleaning unit 80 also move helically when the needle shield 50 is rotated. The cleaning unit which is described in further details in PCT application PCT/EP2019/065451 has a cleaning chamber containing a liquid cleaning agent which is able to clean the distal tip of the needle cannula between injections. In one example, the cleaning agent can be based on the same preservatives as contained in the liquid drug inside the cartridge and in a preferred example; the cleaning agent is the identical same preservative containing liquid drug as present inside the cartridge 5.

When the injection device is delivered to the user, the outwardly pointing protrusion 52 is located in the start of the first track region 60A of the helical track 60 as shown in FIG. 6A.

During the rotation through the first track region 60A (approximately 90°) of the helical track 60 (indicated by the arrow “I” in FIG. 6A), both the needle shield 50 and the needle cannula moves axially such that a distal tip of the needle cannula is maintained inside the cleaning chamber of the cleaning unit 80 as explained in PCT application PCT/EP2019/065451. The movement of the outwardly pointing protrusion 52 through the first 90° inserts the proximal end of the needle cannula into the cartridge 5 and further moves the cartridge 5 a few millimetres in the proximal direction such that a quantum of the liquid drug in the cartridge 5 is forced into the cleaning chamber inside the cleaning unit which is thus filled with liquid drug from the cartridge 5. The preservative of the liquid drug thereafter works as the cleaning agent. The initial movement of the needle shield 50 and the needle cannula is referred to as the initiation of the injection device.

Once the needle shield 50 has been rotated approximately 90°, the axial extension 53 carrying the outwardly pointing protrusion 52 rotationally passes a one-way click-arm 24 provided on the cartridge holder part 20 and thus in the bottom of the helical track 60 as seen in FIG. 6A where after the needle shield 50 cannot be rotated back, i.e. when the axial extension 53 of the needle shield 50 has passed the one-way click-arm 24, the needle cannula has been irreversible inserted into the cartridge 5 and the cleaning chamber has been filled.

In the view shown in FIG. 6A, the first track region 60A of the helical track 60 is visible whereas in the second view in FIG. 6B, the injection device has been rotated to view the second track region 60B of the helical track 60. The cross-over from the first track region 60A of the helical track 60 wherein the injection device is being initiated and to the second track region 60B of the helical track 60 is also indicated by the one-way click arm 24 which in the view of FIG. 6B is hidden below the bridge 35 under which the outwardly pointing protrusion 52 passes once the initiation has been concluded.

As described in further details in WO 2019/002020, the driving force of the torsion spring in the dose engine is released when the user pushes the needle shield 50 against the skin. This axial movement of the needle shield 50 is transferred to an axial movement of a transfer element 90 which transfers the axial movement to the dose engine.

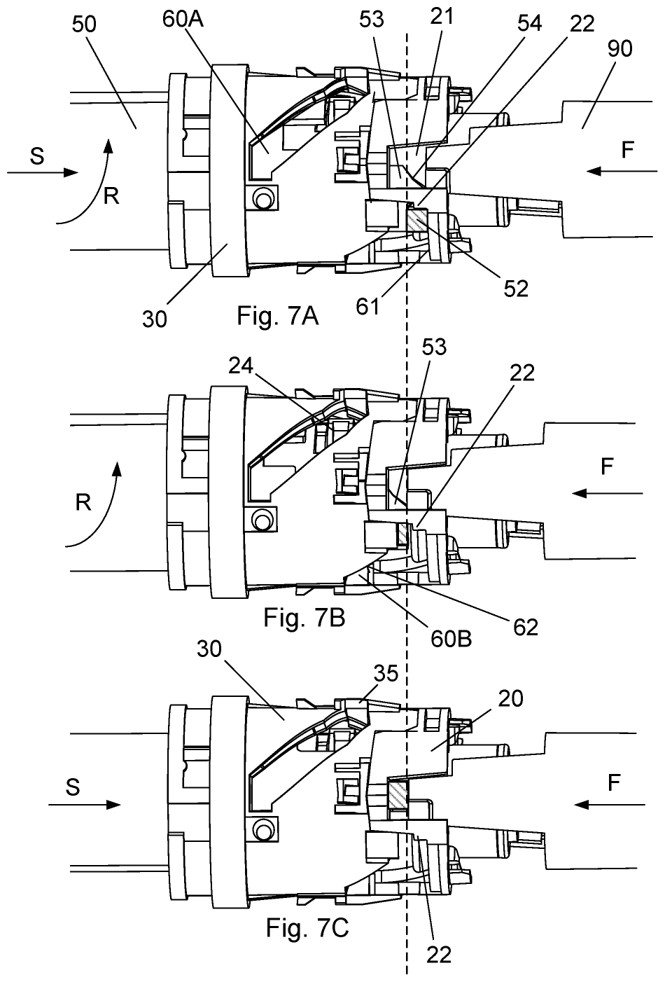

However, when the outwardly pointing protrusion 52 is located in the second track region 60 B it is not possible to move the needle shield 50 axially as the protrusion 52 when moved strictly axially (translational) would encounter and abut against the proximal side wall 61 of the second track region 60B. The user is thus required to rotate the needle shield 50 to an unlocked position (disclosed in FIG. 7C) wherein the outwardly pointing protrusion 52 is able to move axially in the proximal direction.

This is best seen in FIG. 7A-B-C which discloses the cartridge holder part 20 clicked together with the initiation part 30 and with the outwardly pointing protrusion 52 of the needle shield 50 located in the second track region 60B of the helical track 60. The figure also discloses the engagement with the transfer element 90.

In the unlocked position (FIG. 7C), the outwardly pointing protrusion 52 is located in an axial track 21 which connects to the second track region 60B of the helical track 60. The axial track 21 is physically provided in the cartridge holder part 20 as best seen in FIG. 3. Also, in the unlocked position, the outwardly pointing protrusion 52 abuts the transfer element 90 and is able to move the transfer element 90 strictly axially as disclosed in FIG. 7C.

If the user pushes the needle shield 50 against the skin before rotating the needle shield 50 and starts to rotate the needle shield 50 with the needle shield 50 pressed against the skin this will move the outwardly pointing protrusion 52 to abut and follow the proximal side wall 61 and when rotated into the injection position (represented by the axial track 21), the needle shield 50 will suddenly and uncontrolled move in the proximal direction when the outwardly pointing protrusion 52 reaches the axial track 21. This would simultaneously insert the needle cannula into the skin and release the dose engine. However, such sudden insertion and release has the ability to surprise the user who might then react by removing the needle shield 50 and the needle cannula from the skin.

In order to prevent that the user can rotate the outwardly pointing protrusion 52 into the injection position with the needle shield 50 pressed against the skin a physical stop 22 is preferably built into the proximal side wall 61 of the second track region 60B as e.g. disclosed in FIG. 7A-B-C.

In FIGS. 6B and 7A, which disclose the same situation, a force arising from the skin of the user pushes the needle shield 50 in the proximal direction. This force is indicated by the arrow “S” in both FIG. 6B and in FIG. 7A. This force also moves the outwardly pointing protrusion 52 against the proximal wall 61 of the second track region 60B of the helical track 60. However, if the user rotates the needle shield 50 anti-clockwise (seen from a distal position) and towards the unlocked position as indicated by the arrow “R” with the needle shield 50 pressed against the skin, the outwardly pointing protrusion 52 will rotationally engage with the physical stop 22 provided on the proximal wall 61 of the helical track 60 as disclosed in FIG. 7A. This abutment prevents the outwardly pointing protrusion 52 from moving into the axial track 21 and thus prevents a sudden injection in being performed.

As further disclosed in FIG. 7A, the sloped side 54 of the axial extension 53 pushes the transfer element 90 slightly in the proximal direction. The transfer element 90 is biased in the distal direction by a compression force delivered by a non-shown spring. The distal force applied by the spring is indicated by an arrow “F” in FIG. 7A-B-C. The distance that the axial extension 53 moves the transfer element 90 during rotation of the needle shield 50 is not sufficient to cause an injection to be performed.

Due to the physical stop 22, the user is not able to rotational (“R”) move the outwardly pointing protrusion 52 into the axial track 21 while simultaneously pushing the needle shield 50 against the skin (“S”).

In FIG. 7B, the user has removed the needle shield 50 from the skin and the axial force (“S”) arising from the skin is not present anymore. Therefore, the spring force “F” moves the transfer element 90 into the initial position which also moves the outwardly pointing protrusion 52 and the needle shield 50 in the distal direction. Since the needle shield 50 is moved in the distal direction, the axial extension 53 is also moved distally and no force is thus applied to the transfer element 90 in the proximal direction.

The proximal surface of the outwardly pointing protrusion 52 now (FIG. 7B) lies distally to the dotted line “L” and is thus free of the physical stop 22 incorporated in the track. A further rotation of the needle shield 50 and the outwardly pointing protrusion 52 is thus possible in this position.

The rotation of the needle shield 50 (from FIG. 7B to FIG. 7C) brings the outwardly pointing protrusion 52 into the position depicted in FIG. 7C. In this position, the outwardly pointing protrusion 52 rest on the distal side 62 of the helical track 60 and an injection can now be performed by pushing the needle shield 50 against the skin indicated by the arrow “S” in FIG. 7C which moves the outwardly pointing protrusion 52 further into the axial track 21 and henceforth moves the transfer element 90 in the proximal direction to thereby release the torque of the torsion spring and perform an injection.

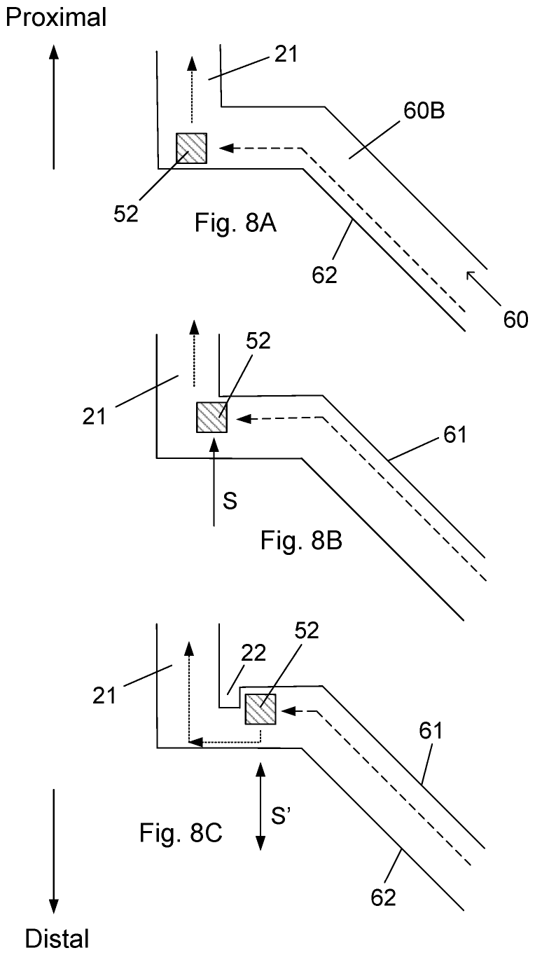

The above is schematically disclosed in FIG. 8A-B-C.

FIG. 8A discloses the prior art way of operation. The user rotates the needle shield 50 e.g. by use of the protective cap 40. This rotation moves the outwardly pointing protrusion 52 along the distal side 62 of the track region 60B. When the end of the track region 60B is reached, the outwardly pointing protrusion 52 is delivered into the axial track 21 and an injection can be performed by pushing the needle shield 50 against the skin such that the outwardly pointing protrusion 52 moves axially through the axial track 21 in the proximal direction.

However, if the user applies a force (“S”) to the needle shield 52 and rotates the needle shield 52 simultaneously therewith the situation disclosed in FIG. 7B occurs.

The outwardly pointing protrusion 52 is pushed against the proximal side 61 of the track region 60B and once the outwardly pointing protrusion 52 is delivered into the axial track 21, the outwardly pointing protrusion 52 and the needle shield 50 will be forced rapidly in the proximal direction such that the needle cannula will penetrate the skin of the user and the dose will be injected almost simultaneously which can be very surprising to the user.

In order to avoid this, a physical stop 22 is built into the proximal side 61 of the track region 60B. This physical stop 22 will prevent the outwardly pointing protrusion 52 from entering into the axial track 21 as disclosed in FIG. 8C.

In order for the outwardly pointing protrusion 52 to pass the physical stop 22, the user needs to remove the needle shield 50 from the skin such that the compression force “F” of the spring via the transfer element 90 can move the needle shield 50 and the outwardly pointing protrusion 52 in the distal direction as indicated by the arrow “S′” in FIG. 8C.

Once the outwardly pointing protrusion 52 rests against the distal side 62 of the track region 60B, the user will be able to rotate the outwardly pointing protrusion 52 into the axial track 21 avoiding the physical stop 22 and thereafter to perform an injection by pushing the needle shield 50 against the skin.

Some preferred embodiments have been shown in the foregoing, but it should be stressed that the invention is not limited to these, but may be embodied in other ways within the subject matter defined in the following claims.

1. A shield trigger mechanism for triggering the ejection of a dose of a liquid drug from a spring driven injection device, comprising:
a needle shield which is rotatably relatively to a housing structure between a locked position and an unlocked position,
wherein the needle shield in the locked position is prevented from axial movement relatively to the housing structure,
and wherein the needle shield in the unlocked position is axially movable relatively to the housing structure in response to an axial force (S) being applied to the needle shield to thereby activate the spring driven injection device to automatically eject the dose of the liquid drug,
and which needle shield is rotational guided from the locked position to the unlocked position by a track arrangement,
wherein the track arrangement is configured to prevent the needle shield in rotation from the locked position to the unlocked position during application of the axial force (S) to the needle shield by having a physical stop incorporated in the track arrangement.

2. The shield trigger mechanism according to claim 1, wherein the track arrangement comprises a helical track region

3. The shield trigger mechanism according to claim 2, wherein the helical track region is associated with the housing structure.

4. The shield trigger mechanism according to claim 2, wherein the needle shield is provided with a protrusion guided in the helical track region.

5. The shield trigger mechanism according to claim 2, wherein the helical track region terminates into an axial track.

6. The shield trigger mechanism according to claim 5, wherein the helical track region or the axial track are provided with a physical stop preferably preventing the protrusion on the needle shield in moving from the helical track region and into the axial track during application of the axial force (S) to the needle shield.

7. The shield trigger mechanism according to claim 6, wherein the needle shield is urged in the distal direction when the force (S) is removed from the needle shield whereby the protrusion is able to escape the physical stop.

8. The spring driven injection device for delivering doses of a liquid drug, comprising
a housing structure having a container containing the liquid drug and a spring driven dose engine,
a needle shield which is rotatably relatively to the housing structure between a locked position and an unlocked position,
and wherein the needle shield when in the locked position is prevented from axial movement relatively to the housing structure,
and wherein the needle shield in the unlocked position is axially movable relatively to the housing structure in response to an axial force being applied to the needle shield to thereby activate the spring driven dose engine to automatically eject the dose of the liquid drug from the container,
and which needle shield is rotational guided from the locked position to the unlocked position in a track arrangement,
wherein the track arrangement is configured to prevent the needle shield in rotation from the locked position to the unlocked position during application of the axial force (S) to the needle shield, by having a physical stop incorporated in the track arrangement.

9. The spring driven injection device according to claim 8, wherein the track arrangement comprises a helical track region.

10. The spring driven injection device according to claim 9, wherein the helical track region is associated with the housing structure.

11. The spring driven injection device according to claim 9 wherein the needle shield is provided with a protrusion guided in the helical track region.

12. The spring driven injection device according to claim 9, wherein the helical track region terminates into an axial track.

13. The spring driven injection device according to claim 12, wherein the helical track region or the axial track are provided with a physical stop preventing the protrusion on the needle shield in moving from the helical track region and into the axial track during application of the axial force (S) to the needle shield.

14. The spring driven injection device according to claim 13, wherein the needle shield is urged in the distal direction when the force (S) is removed from the needle shield whereby the protrusion is able to escape the physical stop.
